(19) United States
(12) Patent Application Publication
(10) Pub. No.: US 2008/0293644 A1
(43) Pub. Date: Nov. 27, 2008

(54) GUAVA EXTRACT

(76) Inventor: Thomas Eidenberger, Steyr (AT)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/106,612

(22) Filed: Apr. 21, 2008

Related U.S. Application Data
(60) Provisional application No. 60/914,418, filed on Apr. 27, 2007.

(51) Int. Cl. A61K 31/704; A61P 9/10; A61P 3/04

(52) U.S. Cl. 514/25

(57) ABSTRACT

The invention describes the preparation, isolation and use of an extract of guava fruit for the treatment of a disease or condition related with, caused by or mediated by dipeptidyl peptidase IV.

### CROSS REFERENCE TO RELATED APPLICATIONS

This application claims benefit of U.S. Ser. No. 60/914,418 filed on Apr. 27, 2007, entitled “Guava Extract” by Thomas Eidenberger, the contents of which are incorporated in their entirety herein by reference.

### FIELD OF THE INVENTION

The invention relates generally to methods to isolate and purify guava extracts and related compositions.

### BACKGROUND OF THE INVENTION

Polyphenols are widely distributed in plants and a major class of secondary plant products. More than 5000 different polyphenols have been identified and the estimated number of polyphenols in nature is most likely much higher than that due the complexity of the class of compounds. In general polyphenols are found in plants as free polyphenol monomers (e.g. flavanols, flavanons, flavons, anthocyanidins) or as conjugated tannins such as hydrolyzable tannins, derived tannins or condensed tannins and proanthocyanidins.

Many health benefits of tea, fruits and vegetables are attributed to polyphenols. For example, green tea polyphenols, are valued due to their proven antioxidative, antimutagenic, anticarcinogenic and hypocholesteremic effects as well as their potential to prevent cardiovascular diseases.

Tea polyphenols are generally well characterised in the literature. It has been reported that 200 ml green tea contains up to 140 mg (−)-epigallocatechin gallate (EGCG), 65 mg (−)-epigallocatechin (EGC), 28 mg (−)-epicatechin gallate (ECG) and 17 mg (−)-epicatechin. The high content of galloyl-units was shown to be correlated with apoptosis induced in prostate and breast cancer cells and the inhibition of the fatty acid synthase enzyme.

Studies in obese humans and patients suffering from diabetes indicate that hypertriglyceridema is strongly associated with carbohydrate intake most likely to be due to conversion of metabolites of the glycolysis to lipids via de novo lipogenesis.

Obesity is caused by the results of an imbalance between energy intake and expenditure. Excess energy is stored in fat cells that enlarge or increase in number. Moreover, obesity is a strong risk factor for various diseases, such as hypertension, hyperlipidemia, arteriosclerosis, hyperglycemia, and diabetes. Therefore, an effective way to prevent obesity is to inhibit fat absorption from intestine.

In recent years, it was found that some diseases associated with hyperglycemia, such as especially diabetes mellitus I and II, are related to the activity of the dipeptidyl peptidase IV (DP-IV) enzyme.

DP-IV is a membrane-associated peptidase of 766 amino acids that is widely distributed in various tissues. DP-IV also exists as a soluble circulating form in plasma. Significant DP-IV activity is detectable in plasma from humans and rodents. The first biological principle of membrane-associated DP-IV relates to intracellular signalling pathways. The second principal biological activity of DP-IV is its enzymatic function in plasma. DP-IV prefers peptidase substrates with an amino-terminal proline or alanine at position 2, but may also cleave substrates with non-preferred amino acids at position 2. Observations from a number of laboratories delineated the importance of DP-IV-mediated inactivation incretins such as glucagon-like peptide-1 amide (“GLP-1”) as a key determinant of GLP-1 bioactivity.

Several DP-IV inhibitors have been characterized, and they appear to lower blood glucose in diabetic rodents via prolongation of GLP-1 and gastric inhibitory polypeptide (“GIP”) action in plasma. Generally such inhibitors are not derived from naturally occurring sources and can have some unwanted side effect(s).

Therefore, a need continues to exist for composition that can help alleviate one or more of these pervasive conditions and/or methods that provide suitable compositions, particularly from naturally occurring sources.

### BRIEF SUMMARY OF THE INVENTION

The present invention surprisingly provides isolated and/or purified guava extracts that have dipeptidyl peptidase IV (DP-IV) inhibitory activity. The guava extracts described herein include polyphenols, such as flavonols. It has now surprisingly been found that guaijaverin, a member of the chemical class of flavonols, is very effective as a DP-IV inhibitor, therefore rendering this compound, as well as the extracts described herein, as suitable for the treatment of diseases associated with DP-IV activity.

The present invention also pertains to methods of preparing the guava extracts described herein.

The present invention further pertains to methods of treatment of various ailments by administration of a therapeutically effective amount of the guava extracts described herein.

The present invention also pertains to pharmaceutical compositions that include the guava extracts described herein and a pharmaceutically acceptable carrier.

Therefore, the present invention further provides bioavailable polyphenolic derivatives in therapeutic levels derived from guava extracts described herein.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE DRAWINGS

FIG. 1 is a dose/response inhibition curve for the synthetic inhibitor P32/98.

FIG. 2 is a dose/response inhibition curve for the isolated guava extract of the invention.

FIG. 3 shows that guaijaverin yields a dose dependent inhibition of DP-IV.

FIG. 4 shows that guaijaverin yields a dose dependent inhibition of DP-IV.

FIG. 5 provides the main quercetin-glycosides found in the isolated guava extract.

FIG. 6 shows the inhibition of DP-IV by different guava extract constituents

### DETAILED DESCRIPTION

Glucose-induced insulin secretion is modulated by a number of hormones and neurotransmitters. Of specific interest are the two gut hormones, glucagon-like peptide-1 (GLP-1) and gastric inhibitory peptide (GIP), both of which are insulinotropic agents. Insulinotropic agents can stimulate, or cause the stimulation of, the synthesis or expression of the hormone insulin.

GLP-1 is a potent intestinal insulinotropic agent that augments insulin secretion and acutely lowers glucose levels, including levels observed in Type I and Type II diabetes. GLP-1 is formed by alternative tissue-specific cleavages in the L cells of the intestine, the alpha-cells of the endocrine pancreas, and neurons in the brain. GIP is synthesized and released from the duodenum and proximal jejunum postprandially. Its release depends upon several factors including meal content and pre-existing health status. It was initially discovered and named for its gastric acid inhibitory properties. However, as research into this hormone has progressed, more relevant physiological roles have been elucidated. Specifically, GIP is an insulinotropic agent with a stimulatory effect on insulin synthesis and release.

Dipeptidyl peptidase IV (DP IV also known as CD 26) is an enzyme that is an exopeptidase that selectively cleaves peptides after penultimate N-terminal proline and alanine residues. Endogenous substrates for this enzyme include the incretins, such as glucose-dependent insulinotropic polypeptides, like GIP and GLP-1. In the presence of DP IV, these hormones are enzymatically reduced to inactive forms. The inactive form of GIP and GLP cannot induce insulin secretion, thus blood glucose levels are elevated, especially in the hyperglycemic state. Elevated blood glucose levels have been associated with much different pathology, including diabetes mellitus (Type 1 and 2) and the sequelae accompanying diabetes mellitus.

It has also been discovered that DP IV plays a role in T-cell-mediated immune responses, for example, in transplantations. Inhibition of DP IV has been demonstrated to prolong cardiac allografts. Additionally, the inhibition of DP IV has contributed to the suppression of rheumatoid arthritis. DP IV has also been attributed a role in HIV's penetration into T-cells (T-helper cells).

Diseases, which characteristically demonstrate hyperglycemia, include diseases such as Diabetes mellitus, Type I and II. Diabetes may generally be characterized as an insufficient hormone output by the pancreatic beta-cells. Normally, these cells synthesize and secrete the hormone insulin. In Type I diabetes, this insufficiency is due to destruction of the beta cells by an autoimmune process. Type II diabetes is primarily due to a combination of beta cell deficiency and peripheral insulin resistance. In the diabetic patient, the number of beta cells is reduced so not only is there a concern regarding the ability of beta cells to synthesize and release physiological insulin, but there is also a concern surrounding the critical mass of these insulin producing pancreatic cells. Loss of beta cells is known to occur with the presence of diabetes. With the loss of these insulin producing cells, there exists a strain on the endocrine function of the pancreas to produce, for example, insulin. With the loss in insulin output, pathological processes due to hyperglycemia can become exacerbated.

Conventional guava extracts, often prepared only from water or alcohols and a guava material, contain sesquiterpenes, tannins, and other components that interfere with the inhibition of DP-IV activity associated with active components as presently disclosed. Surprisingly, the isolation and purification, and concentration of the components by the extraction process of the invention enhances the DP-IV inhibitory effect in a synergistic type fashion, substantially eliminating the unwanted materials such as sesquiterpenes and tannins.

By the term “substantially eliminating” or “substantially removed” is meant that the extract is purified by one of the processes detailed throughout. Contaminants that reduce DP-IV activity are advantageously removed from the concentrate during the process, providing an extract composition that has the majority of the components of the guava material that would interfere with DP-IV activity. Such components include sesquiterpenes and tannins. Generally, the concentrate has less than about 10% of either of both sesquiterpenes or tannins, in particular less than about 5% of either or both of sesquiterpenes or tannins, particularly less than about 1% of either or both of sesquiterpenes or tannins and most particularly less than about 0.5% of either or both of sequiterpenes or tannins, based on the total weight of the extract.

Thus the present invention provides a resultant concentrate (extract) that has a specific content (standardized) of active components. That is, the extract has a content of peltatoside present in an amount of about 5 to about 25 milligrams, quercitin-hexose present in an amount of about 30 to about 75 milligrams, methylquercetin-hexose present in an amount of about 40 to about 70 milligrams, isoquercitrin present in an amount of about 5 to about 40 milligrams, morin-pentose is present in an amount of about 15 to about 45 milligrams, guaijaverin is present in an amount of about 45 to about 80 milligrams and quercetin-pentose is present in an amount of about 60 to about 95 milligrams based on one gram total weight of the extract (dry solid substance).

More specifically, the extract has a standardized content peltatoside present in an amount of about 15 to about 17 milligrams, quercitin-hexose present in an amount of about 50 to about 54 milligrams, methylquercetin-hexose present in an amount of about 50 to about 56 milligrams, isoquercitrin present in an amount of about 18 to about 21 milligrams, morin-pentose is present in an amount of about 26 to about 29 milligrams, guaijaverin is present in an amount of about 63 to about 68 milligrams and quercetin-pentose is present in an amount of about 75 to about 79 milligrams based on one gram total weight of the extract.

In the specification and in the claims, the terms “including” and “comprising” are open-ended terms and should be interpreted to mean “including, but not limited to . . . . ” These terms encompass the more restrictive terms “consisting essentially of” and “consisting of:”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. All publications and patents specifically mentioned herein are incorporated by reference in their entirety for all purposes including describing and disclosing the chemicals, instruments, statistical analyses and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

Various sources of guava fruit are acceptable. Although not to be limited by the following, the guava material can be ideally obtained from plants such as Psidium cattleianum, Psidium cattleianum ssp. Lucidum, Psidium guajava, Psidium guineense, Psidium littorale, Psidium molle or Psidium schiedeanum.

The term “extract” is intended to mean guava extract compositions that are obtained from plant sources, such as leaves, twigs, bark, roots, stem, seeds, flowers, berries, fruit, for example, by isolation methods described herein. It has been surprisingly found that extract of guava material provides an extract that has an enhanced weight percentage of polyphenols such as guaijaverin and quercetin, as compared to guava sources per se or as a freeze dried product. Ideally, the pulp and the juice of the guava fruit are used. When leaves are used, it is often best to pulverize the leaves prior to extraction. Drying the leaves prior to pulverization is optional.

Guaijaverin is known as quercetin-3-O-alpha-L-arabinopyranoside, the arabinosic glycoside of quercetin. Guaijaverin has the formula:

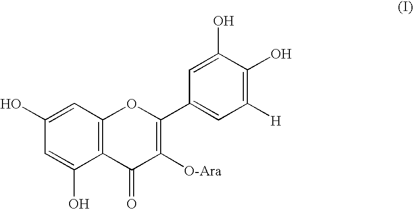

wherein “Ara” is arabinose (O-α-L-arabinopyranose).

More specifically, guaijaverin has a structure such as (Ia)

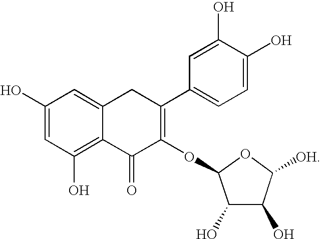

Quercetin (II) is a flavonoid and more specifically a flavonol.

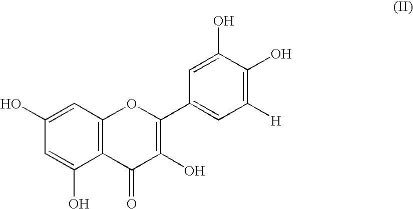

Quercetin is the aglycone form of a number of other flavonoid glycosides, such as rutin, guaijaverin and quercitrin found in citrus fruit. Quercetin has demonstrated significant anti-inflammatory activity because of direct inhibition of several initial processes of inflammation. For example, it inhibits both the manufacture and release of histamine and other allergic/inflammatory mediators. In addition, it exerts potent antioxidant activity and vitamin C-sparing action.

Quercetin forms the glycosides quercitrin and rutin together with rhamnose (quercetin-3-O-rhamnoside, IIa) and rutinose respectively.

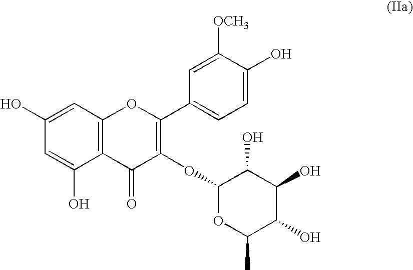

Likewise, quercetin forms the glycoside guaijaverin with arabinose. In other aspects, the enolic-hydroxyl adjacent to the carbonyl of the chromone of formula II can be O-β-D-glucopyranosyl, O-β-D-galactopyranosyl, O-α-L-arabinopyranosyl (guaijaverin) or O-α-L-rhamnopyranosyl.

The present invention pertains to an isolated guava extract that includes quercetin related flavonol-glycosides including peltatoside, quercitin-hexose, methylquercetin-hexose, isoquercitrin, morin-pentose, guaijaverin and quercitrin-pentose. In one aspect, at least about 30 weight percent of the isolated guava extract composition contains flavonol-glycosides based on the total weight of the extract.

In another aspect, of the about 30 weight percent quercetin flavonol-glycosides contained within the isolated guava extract, about 95 percent of the percentage weight is attributable to peltatoside, quercitin-hexose, methylquercetin-hexose, isoquercitrin, morin-pentose, guaijaverin and quercitrin-pentose.

Thus, utilizing Formula III

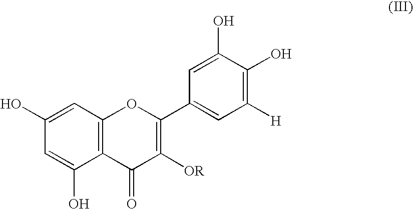

R is defined as follows for:

peltatoside includes an arabinoglucose for R;

quercitin-hexose includes a hexose for R;

guaijaverin includes an arabinose for R;

quercitrin-pentose includes a pentose for R;

wherein the terms hexose and pentose do not denote specific stereochemistry of the glycoside.

For example, peltatoside can have formula (IIIa)

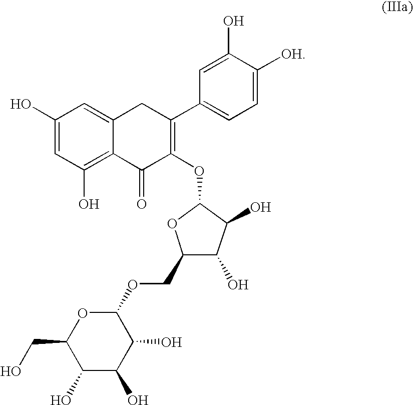

Quercitin-hexose can have the formula (IIIb)

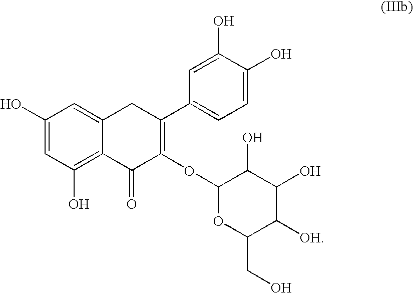

Quercitrin-pentose can have the formula (IIIc)

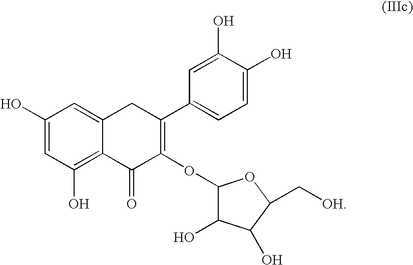

Methylquercetin-hexose can be depicted as Formula IV

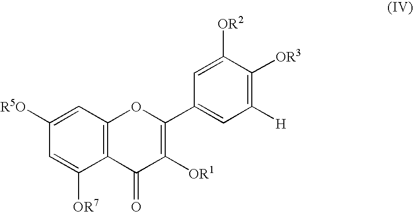

wherein R¹ is a hexose of undefined stereochemistry and either one of R², R³, R⁵ or R⁷ is a methyl group and the others are hydrogen atoms. It is possible that methylquercetin is a mixture of isomers since it is difficult to determine which hydroxyl of R²/R³/R⁵/R⁷ is methylated.

For example, methyl-quercetin-hexose can have the formula (IVa) where R² is a methyl and the remaining R groups are all hydrogen.

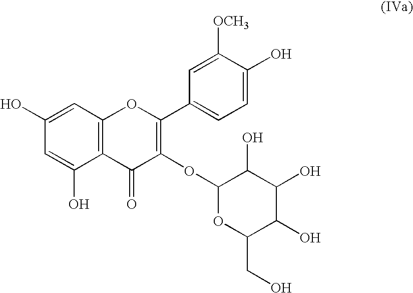

Isoquercitrin can be depicted as Formula V

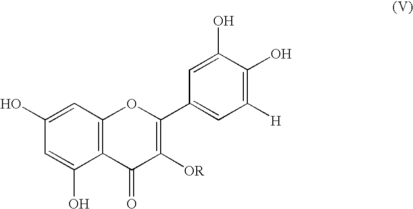

where R is glucose; hence quercetin-3-O-glucoside.

Isoquercitrin can have the formula (Va)

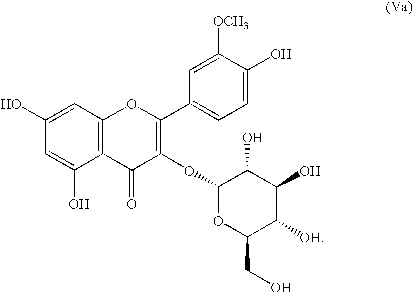

Morin-pentose can be depicted as Formula VI

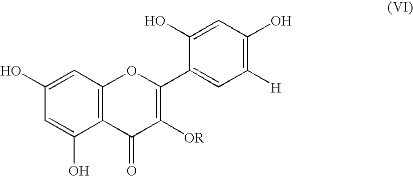

where R is a pentose with undefined stereochemistry.

In one aspect, morin-pentose can be (VIa)

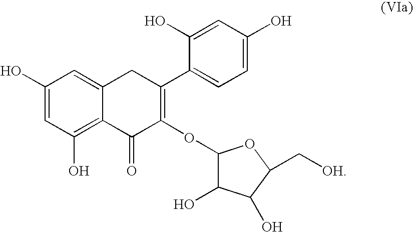

In another aspect of the isolated guava extract of the invention, guaijaverin is greater than about 5 weight percent and peltatoside is greater than about 10 weight percent of the total amount of flavonol-glycosides of the extract.

In still another aspect of the isolated guava extract of the invention, peltatoside is present at about 15 to about 17 weight percent, quercitin-hexose is present at about 50 to about 54 weight percent, methylquercetin-hexose is present at about 50 to about 56 weight percent, isoquercitrin is present at about 18 to about 21 weight percent, morin-pentose is present at about 26 to about 29 weight percent, guaijaverin is present at about 63 and about 68 weight percent and quercetin-pentose is present at about 75 to about 79 weight of the total weight of the extract.

In another aspect, it is possible that the percentage ranges of quercetin-pentose or morin-pentose are reversed.

The guava extract is generally obtained with a solvent selected from aliphatic alcohols, e.g., ethanol; methanol, propanol, butanol, and the like, acetone, hexane, chloroform, ethyl acetate, petroleum ether, mixtures thereof and/or mixtures thereof with water. In particular, an ethanolic extract of guava materials can be prepared with ethanol or with a mixture of ethanol and water. The alcohol to water ratio can be varied from about 99 percent to about 1 percent by weight with all possible percentages in between. In one aspect, the ethanol to water ratio is about 80 percent to about 20 percent by weight.

In one aspect, the extraction of the active components is conducted at an elevated temperature, that is, above room temperature. Generally the extraction mixture is heated between about 30° C. to about 80° C., in particular between about 40° C. and about 70° C., more particularly between about 45° C. and about 65° C., i.e., between about 60° C. and about 62° C. Generally the extraction mixture is heated at the required temperature for a period of between about 1 and about 5 hours, in particular about 2 to about 4 hours and in particular about 2 hours.

In accordance with the present invention, the isolated guava extract contains at least one polyphenolic compound such as guaijaverin and often quercetin.

In one aspect, the isolated guava extract of the present invention provides at least about 30%, 50%, 75% or more, 90% or more and even 95% or more polyphenols. The extract used according to the invention can contain an amount of guaijaverin of about 0.5% by weight or more, more particularly from about 10 to about 50% by weight. Typically, the extract contains an amount of guaijaverin of about 2% by weight to about 4% by weight.

In another aspect, the isolated guava extract of the present invention can contain quercetin.

Oligo- and polymeric polyphenols are differentiated by their stability. Polyphenols, which are hydrolyzed to the basic structures in hot water, are referred to as hydrolyzable tannins. Polyphenols, which are hydrolyzed only in presence of strong acids, are referred to as derived or condensed tannins.

Polyphenols occur in “free” (monomeric) and “conjugated” (oligo-/polymeric) forms.

The present invention provides that the guava extracts of the invention exhibit a higher inhibiting effect towards DP-IV than guava juice or guava concentrates from juice/leaves/stems, etc. Such concentrates simply concentrates where water has been removed. Therefore, guava extracts containing increased amounts of polyphenols, such as guaijaverin and quercetin were found to have a very good inhibiting activity with respect to DP-IV. The unique processing of the guava material as presented throughout the specification provides a unique guava extract with such properties.

The isolated guava extracts of the present invention can be utilized in the treatment of a disease including glucose metabolism disorders, such as obesity, hyperlipidemia, hypertension, arthrosclerosis and diabetes, diabetes mellitus. These diseases have been shown to be linked to the activity of DP-IV.

Furthermore, guaijaverin and/or the guava extract used according to the invention can be used for other therapeutic purposes, such as for lowering LDL cholesterol, as an antioxidant, as an analgesic agent, and as a haemostatic agent, e.g. for relieving conditions associated with women's menstruation.

DP-IV is dipeptidyl protease IV is a membrane-associated peptidase of 766 amino acids that is widely distributed in numerous tissues. DP-IV also exists as a soluble circulating form in plasma as is known in the art.

DP-IV like enzymes includes those that are specific for insulin.

In one exemplary method, the guava extract of the present invention can be isolated by a process generally comprising the steps:

Pulverizing, for example, leaves of Psidium guajava leaves to a fine powder. The leaves can be fresh or dried. In some instances, dried leaves are preferred as they are easier to store.

The powdered guava material is then extracted with ethanol and water at an elevated temperature. The temperature of the extraction solvent is maintained between about 50° C. and about 80° C. The solvent can be a mixture of ethanol and water having a percentage of ethanol to water from about 0 percent (water) to about 95 percent ethanol. Generally, the extraction time period is between about 1 hour and 5 hours at the elevated temperature and a ratio of solvent to powdered guava material of between about 5 and about 10 (weight by weight) is utilized.

Typically the mixture is filtered to remove solids. The filtration step can be accomplished by methods known in the art including gravity filtration, use of microporous filters or membranes or ultrafiltration.

The extraction of the solids can be repeated several times and the filtrates can be combined.

Ultimately, the filtrate(s) is concentrated at reduced pressure until the ethanol is removed to afford a concentrate.

The concentrate is then centrifuged to remove insoluble impurities to obtain a clarified liquid.

The clarified liquid is loaded onto a chromatographic column filled with a macroporous resin. In certain aspects, the macroporous resin is hydrophobic. Impurities are further removed by washing the resin with water. The active components are then desorbed by treating the resin with ethanol and collecting the ethanolic eluate.

The eluate is the concentrated under reduced pressure to yield the guava extract.

Optionally, the guava extract collected after treatment with the resin with water and ethanol washes, can be further treated with a polyamide resin and water to further remove impurities. The materials adsorbed onto the resin can be removed by washing with an alcohol, such as ethanol, and the liquids combined. Concentration under reduced pressure affords a guava extract concentrate.

Optionally, the guava extract concentrate can be further purified by passing the concentrate through a membrane having a porosity of between about 0.2 and about 2 microns, or by ultrafiltration.

It should be understood that one or more of the above steps can be eliminated, repeated, or changed in sequence.

Typically the isolated guava extract is concentrated by various methods to provide a solution enriched in active components such as polyphenols. For example, ultrafiltration can be used to remove unwanted components by molecular weight cut offs. The retentate from the filtration can be stored as a liquid or, for example, can then be further concentrated into a powder by spray drying, freeze drying, flash drying, fluidized bed drying, ring drying, tray drying, vacuum drying, radio frequency drying or microwave drying. Ultimately, the extract should contain at least 10% by weight polyphenolic content. The extracts, therefore, contain polyphenolic derivatives and, optionally, other plant materials such as other flavinoids, sugars, etc. that provide the ability to inhibit DP-IV activity.

The guava extracts can be further purified by one or more methods known in the art, such as chromatography, gel chromatography, high performance liquid chromatography, crystallization, affinity chromatography, partition chromatography and the like. Identification of the particular polyphenol(s) can be accomplished by methods know to those skilled in the art and include ¹H NMR, chemical degradation, chromatography and spectroscopy, especially homo- and heteronuclear two-dimensional NMR techniques for the characterization of the isolated polyphenolic compounds.

The term “purified” or “isolated” is used in reference to the purification and/or isolation of one or more polyphenolic compounds from a guava extract as described above. Again using conventional methods known in the art, various components of the guava extract can be separated into purified materials. In one aspect of the invention, the polyphenol(s) of the extract are substantially purified and isolated by techniques known in the art. The purity of the purified compounds is generally at least about 90%, preferably at least about 95%, and most preferably at least about 99% and even more preferably at least about 99.9% (e.g. about 100%) by weight.

Therefore, the present invention further provides bioavailable isolated guava extract compositions described herein that are useful to treat various afflictions noted herein. The guava extracts can be administered by a number of methods, as discussed infra.

The compositions of the invention can be incorporated into various foods, drinks, snacks, etc. In one aspect, the composition can be sprinkled onto a food product, prior to consumption. If sprinkled onto a food product, a suitable carrier such as starch, sucrose or lactose, can be used to help distribute the concentration of the guava extract making it easier to apply to the food product.

The compositions of the present invention can also be provided as supplements in various prepared food products. For the purposes of this application, prepared food product means any natural, processed, diet or non-diet food product to which a composition of the invention has been added. The compositions of the present invention can be directly incorporated into many prepared diet food products, including, but not limited to diet drinks, diet bars and prepared frozen meals. Furthermore, the compositions of the inventions can be incorporated into many prepared non-diet products, including, but not limited to candy, snack products such as chips, prepared meat products, milk, cheese, yogurt, sport bars, sport drinks, mayonnaise, salad dressing, bread and any other fat or oil containing foods. As used herein, the term “food product” refers to any substance fit for human or animal consumption.

The compositions of the invention can be added to various drinks, such as fruit juices, milkshakes, milk, etc.

Furthermore, guaijaverin and/or the guava extract used according to the invention may be mixed with other plant extracts like e.g., extracts from bitter melon, mulberry leaves, and banaba leaves.

The preferred method of administration is oral. The compositions of the invention can be formulated with suitable carriers such as starch, sucrose or lactose in tablets, capsules, solutions, syrups and emulsions. The tablet or capsule of the present invention can be coated with an enteric coating that dissolves at a pH of about 6.0 to 7.0. A suitable enteric coating, which dissolves in the small intestine but not in the stomach, is cellulose acetate phthalate.

Formulation of the compositions of the invention into a soft gel capsule can be accomplished by many methods known in the art. Often the formulation will include an acceptable carrier, such as an oil, or other suspending or emulsifying agent.

Suitable optional carriers include but are not limited to, for example, fatty acids, esters and salts thereof, that can be derived from any source, including, without limitation, natural or synthetic oils, fats, waxes or combinations thereof. Moreover, the fatty acids can be derived, without limitation, from non-hydrogenated oils, partially hydrogenated oils, fully hydrogenated oils or combinations thereof. Non-limiting exemplary sources of fatty acids (their esters and salts) include seed oil, fish or marine oil, canola oil, vegetable oil, safflower oil, sunflower oil, nasturtium seed oil, mustard seed oil, olive oil, sesame oil, soybean oil, corn oil, peanut oil, cottonseed oil, rice bran oil, babassu nut oil, palm oil, low erucic rapeseed oil, palm kernel oil, lupin oil, coconut oil, flaxseed oil, evening primrose oil, jojoba, wheat germ oil, tallow, beef tallow, butter, chicken fat, lard, dairy butterfat, shea butter or combinations thereof.

Specific non-limiting exemplary fish or marine oil sources include shellfish oil, tuna oil, mackerel oil, salmon oil, menhaden, anchovy, herring, trout, sardines or combinations thereof. In particular, the source of the fatty acids is fish or marine oil (DHA or EPA), soybean oil or flaxseed oil. Alternatively or in combination with one of the above identified carrier, beeswax can be used as a suitable carrier, as well as suspending agents such as silica (silicon dioxide).

The formulations of the invention are also considered to be nutraceuticals. The term “nutraceutical” is recognized in the art and is intended to describe specific chemical compounds found in foods that can prevent disease or ameliorate an undesirable condition.

The formulations of the invention can further include various ingredients to help stabilize, or help promote the bioavailability of the components of the beneficial compositions of the invention or serve as additional nutrients to an individual's diet. Suitable additives can include vitamins and biologically acceptable minerals. Non-limiting examples of vitamins include vitamin A, B vitamins, vitamin C, vitamin D, vitamin E, vitamin K and folic acid. Non-limiting examples of minerals include iron, calcium, magnesium, potassium, copper, chromium, zinc, molybdenum, iodine, boron, selenium, manganese, derivatives thereof or combinations thereof. These vitamins and minerals can be from any source or combination of sources, without limitation. Non-limiting exemplary B vitamins include, without limitation, thiamine, niacinamide, pyridoxine, riboflavin, cyanocobalamin, biotin, pantothenic acid or combinations thereof.

Various additives can be incorporated into the present compositions. Optional additives of the present composition include, without limitation, hyaluronic acid, phospholipids, starches, sugars, fats, antioxidants, amino acids, proteins, flavorings, coloring agents, hydrolyzed starch(es) and derivatives thereof or combinations thereof.

As used herein, the term “antioxidant” is recognized in the art and refers to synthetic or natural substances that prevent or delay the oxidative deterioration of a compound. Exemplary antioxidants include tocopherols, flavonoids, catechins, superoxide dismutase, lecithin, gamma oryzanol; vitamins, such as vitamins A, C (ascorbic acid) and E and beta-carotene; natural components such as camosol, camosic acid and rosmanol found in rosemary and hawthorn extract, proanthocyanidins such as those found in grapeseed or pine bark extract, and green tea extract.

Compositions comprising the guava extract compositions of the invention can be manufactured by methods of conventional mixing, dissolving, granulating, dragee-making levigating, emulsifying, encapsulating, entrapping or lyophilization processes. The compositions can be formulated in conventional manner using one or more physiologically acceptable carriers, diluents, excipients or auxiliaries that facilitate processing of the guava extract compositions into preparations that can be used.

The compositions of the invention can take a form suitable for virtually any mode of administration, including, for example, oral, buccal, systemic, injection, transdermal, rectal, vaginal, etc., or a form suitable for administration by inhalation or insufflation.

Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the guava extract compositions in aqueous or oily vehicles. The compositions can also contain formulating agents, such as suspending, stabilizing and/or dispersing agent. The formulations for injection can be presented in unit dosage form, e.g., in ampoules or in multidose containers, and can contain added preservatives.

Alternatively, the injectable formulation can be provided in powder form for reconstitution with a suitable vehicle, including but not limited to sterile pyrogen free water, buffer, dextrose solution, etc., before use. To this end, the guava extract compositions can be dried by any art-known technique, such as lyophilization, and reconstituted prior to use.

For transmucosal administration, penetrants appropriate to the barrier to be permeated are used in the formulation. Such penetrants are known in the art.

For oral administration, the compositions of the invention can take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets can be coated by methods well known in the art with, for example, sugars, films or enteric coatings.

Liquid preparations for oral administration can take the form of, for example, elixirs, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non aqueous vehicles (e.g., almond oil, oily esters, ethyl alcohol, or fractionated vegetable oils); and preservatives (e.g., methyl or propyl p hydroxybenzoates or sorbic acid). The preparations can also contain buffer salts, preservatives, flavoring, coloring and sweetening agents as appropriate.

Preparations for oral administration can be suitably formulated to give controlled release of the guava extract composition as is well known. For buccal administration, the compositions can take the form of tablets or lozenges formulated in conventional manner.

For rectal and vaginal routes of administration, the guava extract compositions can be formulated as solutions (for retention enemas) suppositories or ointments containing conventional suppository bases such as cocoa butter or other glycerides.

For nasal administration or administration by inhalation or insufflation, the guava extract compositions can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit can be determined by providing a valve to deliver a metered amount. Capsules and cartridges for use in an inhaler or insufflator (for example capsules and cartridges comprised of gelatin) can be formulated containing a powder mix of the compound and a suitable powder base such as lactose or starch.

For prolonged delivery, the guava extract compositions can be formulated as a depot preparation for administration by implantation or intramuscular injection. The guava extract compositions can be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch, which slowly releases the guava extract compositions for percutaneous absorption, can be used. To this end, permeation enhancers can be used to facilitate transdermal penetration of the guava extract compositions. Suitable transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other delivery systems can be employed. Liposomes and emulsions are well-known examples of delivery vehicles that can be used to deliver guava extract compositions. Certain organic solvents such as dimethylsulfoxide (DMSO) can also be employed, although usually at the cost of greater toxicity.

The compositions can, if desired, be presented in a pack or dispenser device, which can contain one or more unit dosage forms containing the guava extract compositions. The pack can, for example, comprise metal or plastic foil, such as a blister pack. The pack or dispenser device can be accompanied by instructions for administration.

Soft gel or soft gelatin capsules can be prepared, for example, without limitation, by dispersing the formulation in an appropriate vehicle (e.g., rice bran oil, and/or beeswax) to form a high viscosity mixture. This mixture is then encapsulated with a gelatin based film using technology and machinery known to those in the soft gel industry. The capsules so formed are then dried to constant weight. Typically, the weight of the capsule is between about 100 to about 2500 milligrams and in particular weigh between about 1500 and about 1900 milligrams, and more specifically can weigh between about 1500 and about 2000 milligrams.

For example, when preparing soft gelatin shells, the shell can include between about 20 to 70 percent gelatin, generally a plasticizer and about 5 to about 60% by weight sorbitol. The filling of the soft gelatin capsule is liquid (principally a carrier such as rice bran oil or wheat germ oil and/or beeswax if desired) and can include, apart from the guava extract compositions, a hydrophilic matrix. The hydrophilic matrix, if present, is a polyethylene glycol having an average molecular weight of from about 200 to 1000. Further ingredients are optionally thickening agents and/or emulsifying agent(s). In one embodiment, the hydrophilic matrix includes polyethylene glycol having an average molecular weight of from about 200 to 1000, 5 to 15% glycerol, and 5 to 15% by weight of water. The polyethylene glycol can also be mixed with propylene glycol and/or propylene carbonate.

In another embodiment, the soft gel capsule is prepared from gelatin, glycerine, water and various additives. Typically, the percentage (by weight) of the gelatin is between about 30 and about 50 weight percent, in particular between about 35 and about weight percent and more specifically about 42 weight percent. The formulation includes between about 15 and about 25 weight percent glycerine, more particularly between about 17 and about 23 weight percent and more specifically about 20 weight percent glycerine.

The remaining portion of the capsule is typically water. The amount varies from between about 25 weigh percent and about 40 weight percent, more particularly between about 30 and about 35 weight percent, and more specifically about 35 weight percent. The remainder of the capsule can vary, generally, between about 2 and about 10 weight percent composed of a flavoring agent(s), sugar, coloring agent(s), etc. or combination thereof. After the capsule is processed, the water content of the final capsule is often between about 5 and about 10 weight percent, more particularly 7 and about 12 weight percent, and more specifically between about 9 and about 10 weight percent.

As for the manufacturing, it is contemplated that standard soft shell gelatin capsule manufacturing techniques can be used to prepare the soft-shell product. Examples of useful manufacturing techniques are the plate process, the rotary die process pioneered by R. P. Scherer, the process using the Norton capsule machine, and the Accogel machine and process developed by Lederle. Each of these processes is mature technologies and is all widely available to any one wishing to prepare soft gelatin capsules.

Emulsifying agents can be used to help solubilize the ingredients within the soft gelatin capsule. Specific examples of the surfactant, emulsifier, or effervescent agent include D-sorbitol, ethanol, carrageenan, carboxyvinyl polymer, carmellose sodium, guar gum, glycerol, glycerol fatty acid ester, cholesterol, white beeswax, dioctyl sodium sulfosuccinate, sucrose fatty acid ester, stearyl alcohol, stearic acid, polyoxyl 40 stearate, sorbitan sesquioleate, cetanol, gelatin, sorbitan fatty acid ester, talc, sorbitan trioleate, paraffin, potato starch, hydroxypropyl cellulose, propylene glycol, propylene glycol fatty acid ester, pectin, polyoxyethylene (105) polyoxypropylene (5) glycol, polyoxyethylene (160) polyoxypropylene (30) glycol, polyoxyethylene hydrogenated castor oil, polyoxyethylene hydrogenated castor oil 40, polyoxyethylene hydrogenated castor oil 60, polyoxyl 35 castor oil, polysorbate 20, polysorbate 60, polysorbate 80, macrogol 400, octyldodecyl myristate, methyl cellulose, sorbitan monooleate, glycerol monostearate, sorbitan monopalmitate, sorbitan monolaurate, lauryl dimethylamine oxide solution, sodium lauryl sulfate, lauromacrogol, dry sodium carbonate, tartaric acid, sodium hydroxide, purified soybean lecithin, soybean lecithin, potassium carbonate, sodium hydrogen carbonate, medium-chain triglyceride, citric anhydride, cotton seed oil-soybean oil mixture, and liquid paraffin.

The present invention also provides packaged formulations of the compositions of the invention and instructions for use of the product for appropriate condition(s). Typically, the packaged formulation, in whatever form, is administered to an individual in need thereof. Typically, the dosage requirement is between about 1 to about 4 dosages a day.

Although the present invention describes the preparation, use, manufacture and packaging of the compositions of the invention in soft gelatin capsules for treatment of various conditions, it should not be considered limited to only soft gelatin capsules. Ingestible compositions of the invention can be delivered in traditional tablets, pills, lozenges, elixirs, emulsions, hard capsules, liquids, suspensions, etc. as described above.

The guava extract compositions of the invention, or compositions thereof, will generally be used in an amount effective to achieve the intended result, for example in an amount effective to treat or prevent the particular related condition being treated. The composition can be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient can still be afflicted with the underlying disorder. For example, administration of a composition of the invention to a patient suffering from pain provides therapeutic benefit not only when the underlying condition is eradicated or ameliorated, but also when the patient reports a decrease in the severity or duration of the physical discomfort associated with the pain.

For prophylactic administration, the composition can be administered to a patient at risk of developing one of the previously described conditions.

The amount of composition administered will depend upon a variety of factors, including, for example, the particular indication being treated, the mode of administration, whether the desired benefit is prophylactic or therapeutic, the severity of the indication being treated and the age and weight of the patient, etc. Determination of an effective dosage is well within the capabilities of those skilled in the art.

Total dosage amounts of a guava extract composition will typically be in the range of from about 0.0001 or 0.001 or 0.01 mg/kg/day to about 100 mg/kg/day, but may be higher or lower, depending upon, among other factors, the activity of the components, its bioavailability, the mode of administration and various factors discussed above. Dosage amount and interval can be adjusted individually to provide plasma levels of the compound(s), which are sufficient to maintain therapeutic or prophylactic effect. For example, the compounds can be administered once per week, several times per week (e.g., every other day), once per day or multiple times per day, depending upon, among other things, the mode of administration, the specific indication being treated and the judgment of the prescribing physician. Skilled artisans will be able to optimize effective local dosages without undue experimentation.

The following paragraphs enumerated consecutively from 1 through 56 provide for various aspects of the present invention. In one embodiment, in a first paragraph (1), the present invention provides an isolated guava extract comprising at least about 30 weight percent of quercetin related flavonol-glycosides comprising peltatoside, quercitin-hexose, methylquercetin-hexose, isoquercitrin, morin-pentose, guaijaverin and quercitrin-pentose of the total weight of the extract.

2. The isolated guava extract of paragraph 1, wherein the quercetin flavonol-glycosides comprise 95 weight percent of the total weight of the extract.

3. The isolated guava extract of paragraph 1, wherein guaijaverin is greater than about 5 weight percent and peltatoside is greater than about 10 weight percent of the total amount of flavonol-glycosides of the extract.

4. An isolated guava extract, comprising peltatoside present in an amount of about 15 to about 17 milligrams, quercitin-hexose present in an amount of about 50 to about 54 milligrams, methylquercetin-hexose present in an amount of about 50 to about 56 milligrams, isoquercitrin present in an amount of about 18 to about 21 milligrams, morin-pentose is present in an amount of about 26 to about 29 milligrams, guaijaverin is present in an amount of about 63 to about 68 milligrams and quercetin-pentose is present in an amount of about 75 to about 79 milligrams based on one gram total weight of the extract.

5. An isolated guava extract obtained from a process comprising extracting guava leaves and/or guava fruit with an alcohol and water at an elevated temperature to obtain a resultant extract; subjecting the resultant extract to reduced pressure to produce an aqueous concentrate; subjecting the concentrate to centrifugation to separate solids; subjecting the clarified solution to a chromatography column and eluting the column with water followed by an alcohol to afford an effluent; subjecting the effluent to reduced pressure to produce a second aqueous concentrate; passing the second aqueous concentrate through a polyamide resin to remove impurities and then desorbing active components with an alcohol; concentrating the active components at reduced pressure to afford the isolated extract.

6. The isolated guava extract according to any of paragraphs 1 through 5, which is capable of inhibiting DP-IV activity or DP-IV like activity.

7. A food or beverage comprising the isolated guava extract according to any of paragraphs 1 through 5.

8. A method for producing a diet food or a diet beverage, which comprises: adding the isolated guava extract according to any of paragraphs 1 through 5 to a food or beverage.

9. A dieting method comprising consuming the food, the beverage, or both the food and beverage as recited in paragraph 8.

10. A dipeptidy peptidase IV (DP-IV) inhibitor obtained from a process comprising extracting guava leaves and/or guava fruit with an alcohol and water at an elevated temperature to obtain a resultant extract; subjecting the resultant extract to reduced pressure to produce an aqueous concentrate; subjecting the concentrate to centrifugation to separate solids; subjecting the clarified solution to a chromatography column and eluting the column with water followed by an alcohol to afford an effluent; subjecting the effluent to reduced pressure to produce a second aqueous concentrate; passing the second aqueous concentrate through a polyamide resin to remove impurities and then desorbing active components with an alcohol; concentrating the active components at reduced pressure to afford the DP-IV inhibitor.

11. The DP-IV inhibitor according to paragraph 10, which is capable of inhibiting DP-IV activity or DP-IV like activity.

12. A food or beverage comprising the DP-IV inhibitor according to either of paragraphs 10 or 11.

13. A method for producing a diet food or a diet beverage, which comprises: adding the DP-IV inhibitor according to either of paragraphs 10 or 11 to a food or beverage.

14. A dieting method comprising consuming the food, the beverage, or both the food and beverage as recited in paragraph 13.

15. A method to produce a guava extract capable of inhibiting dipeptidyl peptidase IV activity comprising the steps of:

contacting a guava source of guava leaves and/or guava fruit with an alcohol and water at an elevated temperature to obtain a resultant extract;

subjecting the resultant extract to reduced pressure to produce an aqueous concentrate;

subjecting the aqueous concentrate to centrifugation to separate solids to produce a clarified solution;

subjecting the clarified solution to column chromatography and eluting the column with water followed by elution an alcohol to afford an effluent;

subjecting the effluent to reduced pressure to produce a second aqueous concentrate;

passing the second aqueous concentrate through a polyamide resin to remove impurities and then desorbing active components with an alcohol; and concentrating the active components at reduced pressure to afford a concentrate.

16. The method of paragraph 15, wherein the guava source is guava leaves.

17. The method of either of paragraphs 15 or 16, wherein the alcohol is ethanol.

18. The method of any of paragraphs 15 through 17, wherein the concentration of alcohol is about 80% by volume.

19. The method of any of paragraphs 15 through 18, wherein the ratio of the final volume of alcohol volume to the guava source weight is about 8 to 1.

20. The method of any of paragraphs 15 through 19, wherein the guava source is from a plant selected from Psidium cattleianum, Psidium cattleianum ssp. Lucidum, Psidium guajava, Psidium guineense, Psidium littorale, Psidium molle or Psidium schiedeanum.

21. The method of any of paragraphs 15 through 20, wherein the elevated temperature is between about 40° C. and about 70° C.

22. The method of any of paragraphs 15 through 21, wherein the hydrophobic column is composed of a crosslinked polystyrene/divinylbenzene copolymer.

23. The method of any of paragraphs 15 through 22, wherein the alcohol is ethanol.

24. The method of any of paragraphs 15 through 23, wherein the concentrate is further processed by membrane filtration.

25. The method of any of paragraphs 15 through 24, wherein the concentrate is further subjected to extraction with water at an elevated temperature to afford an aqueous solution;
subjecting the aqueous solution to centrifugation to provide a clarified solution; and
filtering the clarified solution.

26. The method of paragraph 25, wherein the clarified solution is concentrated under reduced pressure.

27. The extract of any of paragraphs 15 through 26.

28. A method to treat a disease or condition which is a glucose metabolism disorder, diabetes mellitus, obesity, atherosclerosis, lowering LDL cholesterol, or for relieving conditions associated with women's menstruation comprising the step of administering to an individual in need thereof, a therapeutically effective amount of any of the compositions of any of paragraphs 1 through 27, such that the disease or condition is treated.

29. A pharmaceutical comprising any of the compositions of paragraphs 1 through 27 and pharmaceutically acceptable salts or esters thereof; and
a pharmaceutically acceptable carrier.

30. A packaged pharmaceutical, suitable to treat a disease or condition which is a glucose metabolism disorder, diabetes mellitus, obesity, atherosclerosis, lowering LDL cholesterol, or for relieving conditions associated with women's menstruation comprising:

the composition of any of paragraphs 1 through 27, and

instructions to treat the disease or condition.

31. An isolated guava extract comprising quercetin related flavonol-glycosides comprising peltatoside and guaijaverin.

32. The isolated guava extract of paragraph 31, further comprising isoquercetin.

33. The isolated guava extract of paragraph 31, wherein sesquiterpenes and tannins are substantially removed.

34. The isolated guava extract of paragraph 33, wherein less than about 5% by weight of the total weight of the extract is sesquiterpenes and tannins.

35. The isolated guava extract of paragraph 34, wherein less than about 1% by weight of the total weight of the extract is sesquiterpenes and tannins.

36. The isolated guava extract of paragraph 31, wherein the quercetin related flavonol glycosides comprise at least 30 weight percent of the total weight of the extract.

37. The isolated guava extract of paragraph 32, wherein sesquiterpenes and tannins are substantially removed.

38. The isolated guava extract of paragraph 37, wherein less than about 5% by weight of the total weight of the extract is sesquiterpenes and tannins.

39. The isolated guava extract of paragraph 38, wherein less than about 1% by weight of the total weight of the extract is sesquiterpenes and tannins.

40. The isolated guava extract of paragraph 32, wherein the quercetin related flavonol glycosides comprise at least 30 weight percent of the total weight of the extract.

41. The isolated guava extract of paragraph 31, further comprising at least one of quercitin-hexose, methylquercetin-hexose, isoquercitrin, morin-pentose, or quercitrin-pentose.

42. The isolated guava extract of paragraph 41, wherein sesquiterpenes and tannins are substantially removed.

13. The isolated guava extract of paragraph 42, wherein less than about 5% by weight of the total weight of the extract is sesquiterpenes and tannins.

44. The isolated guava extract of paragraph 43, wherein less than about 1% by weight of the total weight of the extract is sesquiterpenes and tannins.

45. The isolated guava extract of paragraph 41, wherein the quercetin related flavonol glycosides comprise at least 30 weight percent of the total weight of the extract.

46. The isolated guava extract of paragraph 32, further comprising at least one of quercitin-hexose, methylquercetin-hexose, isoquercitrin, morin-pentose, or quercitrin-pentose.

47. The isolated guava extract of paragraph 46, wherein sesquiterpenes and tannins are substantially removed.

48. The isolated guava extract of paragraph 47, wherein less than about 5% by weight of the total weight of the extract is sesquiterpenes and tannins.

49. The isolated guava extract of paragraph 48, wherein less than about 1% by weight of the total weight of the extract is sesquiterpenes and tannins.

50. The isolated guava extract of paragraph 46, wherein the quercetin related flavonol glycosides comprise at least 30 weight percent of the total weight of the extract.

51. The isolated guava extract according to any of paragraphs 1 through 50, which is capable of inhibiting DP-IV activity or DP-IV like activity.

52. The method of paragraph 28, wherein the composition further comprises one or more of isoquercetin, quercitin-hexose, methylquercetin-hexose, morin-pentose, or quercitrin-pentose.

53. The method of paragraph 52, wherein sesquiterpenes and tannins are substantially removed from the extract.

54. The method of paragraph 53, wherein less than about 5% by weight of the total weight of the extract is sesquiterpenes and tannins.

55. The method of paragraph 52, wherein sesquiterpenes and tannins are substantially removed from the extract.

56. The method of paragraph 55, wherein less than about 5% by weight of the total weight of the extract is sesquiterpenes and tannins.

The following examples are not to be meant as limiting but are presented to provide additional information and support for the invention.

### EXAMPLES

### Example 1

### General Description of the Preparation of an Ethanolic Extract from Psidium guajava

The production process of preparing an extract from the leaves of Psidium guajava is described by following process steps:
Pulverize Psidium guajava's leaves to a fine powder.
Extract with ethanol.
Reduce the pressure of extract liquid and concentrate until evaporation of ethanol.
Eliminate insoluble impurities by centrifugation and obtain a clarified liquid.
Load the clarified liquid onto a chromatographic column filled with a macroporous resin, remove further impurities with water and then desorb active components with ethanol and collect the ethanolic eluate.
Dry the ethanolic eluate to yield Psidium guajava extract.

Details of Extraction:

1. Extract: Powdered Psidium guajava leaves were extracted two times with 80% ethanol at 60±2° C. The extraction duration was 2 hours for each step. The ratio of the final ethanol volume to the raw material powder weight was 8 to 1.

2. Concentration: The ethanolic/aqueous extract was subjected to reduced pressure and concentrated at 60±2° C. until the ethanol was removed affording an aqueous liquid. The vacuum was less than −0.08 MPa.

3. Chromatography: The aqueous liquid was centrifuged to remove any solid(s). The clarified liquid obtained from the centrifuge was loaded onto a macroporous resin. The resin was rinsed with water. Elution of the active components was carried out with 95% ethanol until the liquid was clarified and the color was slightly yellow. The velocity of flow was controlled at 15-20 ml/min. The ethanolic fractions were collected.

4. Drying: The combined ethanolic fractions were subjected to reduced pressure and the eluate was concentrated/dried. The vacuum was less than −0.08 MPa. A powder was obtained.

### Example 2

### Production of Psidium guajava Extract

500 g of pulverized raw material (fresh Psidium guajava fruit) were weighed and transferred into 2000 ml 80% ethanol in a 3-neck flask. The mixture was mixed gently for 2 hours at 60° C. The solution was filtered. The filtrate was collected, and the extraction of the solids was repeated with 2000 ml 80% ethanol under the same conditions. The solution was again filtered, and both filtrates were combined.

The combined filtrate was concentrated under reduced pressure at 60° C. until no ethanol was left. The vacuum was about −0.09 MPa. The resulting ethanol-free liquid was centrifuged to remove solid particles. The liquid was removed from a pellet of solids. 200 ml water was added to the pellet that resulted from centrifugation and the mixture was again centrifuged. Both aqueous supernatants were combined. The clarified liquid was loaded onto a macroporous resin (Type Amberlite XAD4) and rinsed first with 800 ml water at a flow rate of 17 ml/min to wash out water soluble components. The resin was then treated with 1000 ml 95% ethanol at a flow rate of 8.5 ml/min for desorption of the active components and the eluate was collected for 2 hours. The eluate was concentrated at 60° C. under reduced the pressure, followed by drying for 5 hours.

1.4 g Psidium guajava extract was obtained. The content of quercetin was determined to be 0.10%, and the content of guaijaverin was determined to be 0.657% by HPLC on a weight basis.

### Example 3

### Production of Psidium guajava Extract

20 kg pulverized Psidium guajava leaves powder were weighed and put into a 250 L boiler, then 160 L 80% ethanol were added, and the mixture was mixed for 2 hours at 60° C. The solution was filtered and collected. 160 L 80% ethanol was added to the residue. The mixture was extracted for 2 hours at 60° C., filtered, and filtrates were combined. The resulting mixture was concentrated under reduced pressure at 60° C. until there was a trace amount of ethanol remaining. The vacuum during concentration was about −0.09 MPa.

The aqueous mixture was centrifuged. The resulting supernatant was removed from the solid pellet that was formed during centrifugation. The pellet was disrupted, 40 L of water were added to the material and the aqueous mixture was centrifuged. The supernatants from the first extraction and second extraction were combined. The combined aqueous liquids were passed through a macrocrosslinked macroporous resin (Type Amberlite XAD4). First, 160 L water was used to wash the resin after absorption, by which part of the impurities can be eliminated. Then, 350 L 80% ethanol was passed through the resin to elute the active components from the resin. A yellow-brown liquid was collected. The liquid was concentrated under reduced pressure at 60° C. Afterwards it was dried in a vacuum dryer for 5 hours.

1.8 kg Psidium guajava extract was obtained. The content of guaijaverin was 12.44% with 1.02% quercetin as determined by HPLC analysis. (chromatographic column: C18 ODS (Octadecylsilylated); mobile phase: Methanol-1% Glacial Acetic Acid (32:68, v/v) at a wavelength of 360 nm)

STEP B The above product was further concentrated. The product was dissolved in 200 L water. The solution was separated through a polyamide resin (a polyamide 6 resin from Messrs. Sorbent Technologies, Inc.): First, 40 L water was used to wash the resin to remove impurities. Then, 60 L 80% ethanol were used to desorb desired active components. A yellow-brown liquid was collected as the eluent. The eluent was concentrated under reduced pressure at 60° C. and then dried in a vacuum dryer for 5 hours.

0.45 kg Psidium guajava extract was obtained. The content of guaijaverin was 42.09% with 0.96% quercetin as determined by HPLC analysis (as above).

### Example 4

### Investigation of the DP-IV Inhibiting Effect of a Psidium guajava Extract

Methods:

DP-IV activity was measured by a colorimetric assay.

Gly-Pro-4-NA (G0513, Sigma, St. Louis, Mo.), a (synthetic) chromogenic substrate of DP-IV, is hydrolyzed by DP-IV into the dipeptide glycine-proline and 4-nitroaniline, whose rate of appearance was followed quantitatively at 405 nm.

400 μL assay buffer (9.5 g HEPES/l distilled water, pH adjusted to 7.0, product number H4034, Sigma, St. Louis, Mo.), 150 μL human plasma and 100 μL inhibitor solution (or solvent) were transferred into a photometer cuvette, gently mixed and pre-incubated at 37° C. for 3 minutes. The assay was then started by addition of 70 μL substrate solution (8.6 mg Gly-Pro-4-NA in 10 ml assay buffer). The change in absorption at 405 nm was recorded over a period of 20 min.

DP-IV activity is expressed as the linear change in optical density over 20 min (Δ Abs/min).

Sample Preparation:

A Psidium gujava extract obtained according to Example 2, was dissolved at 45° C. for 24 hours in distilled water under stirring conditions. Thereafter the extract was cleared by centrifugation (15,000 rpm, 15 min.), filtration (syringe filter, 0.45 μM), appropriately diluted and submitted to the test assay.

The concentration of the extract was 5 g powder/100 ml water. Dilutions were prepared from the cleared extract by addition of water.

For comparison purposes, DP-IV was inhibited by P32/98 (3N-[(2S,3S)-2-amino-3-methyl-pentanoyl]-1,3-thiazolidine hemifumarate), a synthetic enzyme inhibitor.

A stock solution of 1.60 mg P32/98/ml assay buffer was prepared and diluted with assay buffer to yield concentrations between 0.50 mg/ml and 0.05 mg/ml. 100 μl of these solutions were added to the assay as “inhibitor” solution.

Comparison experiments were carried out in the same test assay system as described further below in the “Results” section.

Data Evaluation:

Results are expressed as %-inhibition derived from the comparison of test results obtained in samples with no inhibitor added to results obtained in samples with added inhibitors or Psidium guajava extract (Example 2) (both in different concentrations

No inhibition (0%) in a test sample indicated the same increase in absorption compared with a sample with no inhibitor added. Full inhibition (100%) indicated no apparent increase in absorption.

All test results were the average of 2 samples. The relative standard deviation of these replicate samples was always less than 7%.

Results:

The assay was calibrated using well known routine procedures:

The pH-optimum of the test assay system was shown to be in the range between pH=6.0 and pH=8.0. An assay temperature range between 32 and 42° C. does not significantly affect the enzyme activity. Any substrate concentration between 5 and 10 μg/10 ml yielded maximum enzyme activity. At the substrate concentration chosen, the increase in absorption was shown to be linear up to 45 minutes. Under the assay conditions chosen, plasma volumes between 100 and 200 μl were shown to yield a dose-dependent, parallel shift of the increase in absorption.

Replicate tests under the final test assay conditions yield a relative standard deviation of less than 7%.

Inhibition of the enzyme by well known unspecific enzyme inhibitors and various solvents produced results shown in Table 1.

TABLE 1
Effect of unspecific enzyme inhibitor on DP-IV activity

| Enzyme Inhibitors (100 mM) | %-Inhibition DP-IV |
|---|---|
| Ethylendiamine•HCl | 19.0 |
| EDTA | 24.0 |
| Thimerosal | <1.0 |
| Methimazol | 10.0 |
| Mercaptoethanol | 15.3 |
| Zinc⁺⁺ | 19.3 |
| Ethanol | 52.8 |
| Methanol | 59.7 |
| DMSO | 75.3 |

As seen, the enzyme DP-IV is not substantially blocked by the unspecific enzyme inhibitors chosen. Mentionable inhibition was achieved by organic solvents. Due to these results, the extracts at hand were dissolved in water, as organic solvents were shown to block the enzyme activity; hence introduction of those solvents would have led to uninterpretable results.

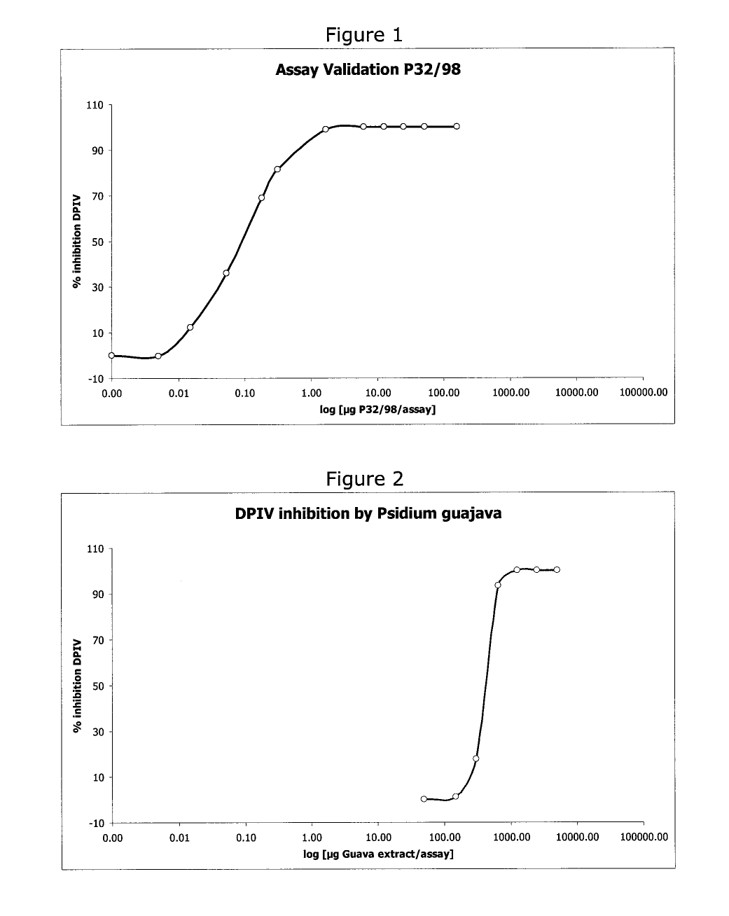

The results of the tests carried out with synthetic inhibitor P 32/98 and Psidium guajava extract are shown in FIGS. 1 and 2, with the concentration of the respective inhibitor plotted on the abscissa, and the respective observed inhibition of DP-IV plotted on the ordinate.

As shown in FIG. 1, the synthetic inhibitor P32/98 yielded a smooth dose/response inhibition curve. In the test assay system chosen, a concentration of approximately 0.10 μg/assay volume yielded a DP-IV inhibition of around 50%.

As shown in FIG. 2, the extract of Psidium guajava also yielded a smooth dose/response inhibition curve. In the test assay system chosen, a concentration between 100-1,000 μg/assay volume yielded a DP-IV inhibition of around 50%.

Hence, Psidium guajava extract was shown to inhibit DP-IV substantially. The difference in potency between Psidium guajava and the synthetic inhibitor P 32/98 amounts to approximately 1,000.

### Example 5

### Investigation of the DP-IV Inhibiting Effect of Guaijaverin

Unless indicated otherwise in the following, the materials and methods applied were the same as those of Example 4.

Sample Preparation:

Guaijaverin was dissolved in HEPES buffer (20 min. ultrasonication followed by shaking for 2 hours at room temperature), appropriately diluted and submitted to the test assay. Dilutions were prepared by addition of HEPES buffer. The concentrations tested were between 70-280 μg/ml test assay.

Data Evaluation:

Results are expressed as %-activity derived from the comparison of test results obtained in positive control samples (no inhibitor added) to results obtained in samples with added guaijaverin at different concentrations.

100% activity in a test sample indicated the same increase in absorption compared in a sample with no inhibitor. Zero activity (0%) indicated no apparent increase in absorption.

All test results represent the average of 2 samples. The relative standard deviation of these replicate samples was always less than 5%.

Results:

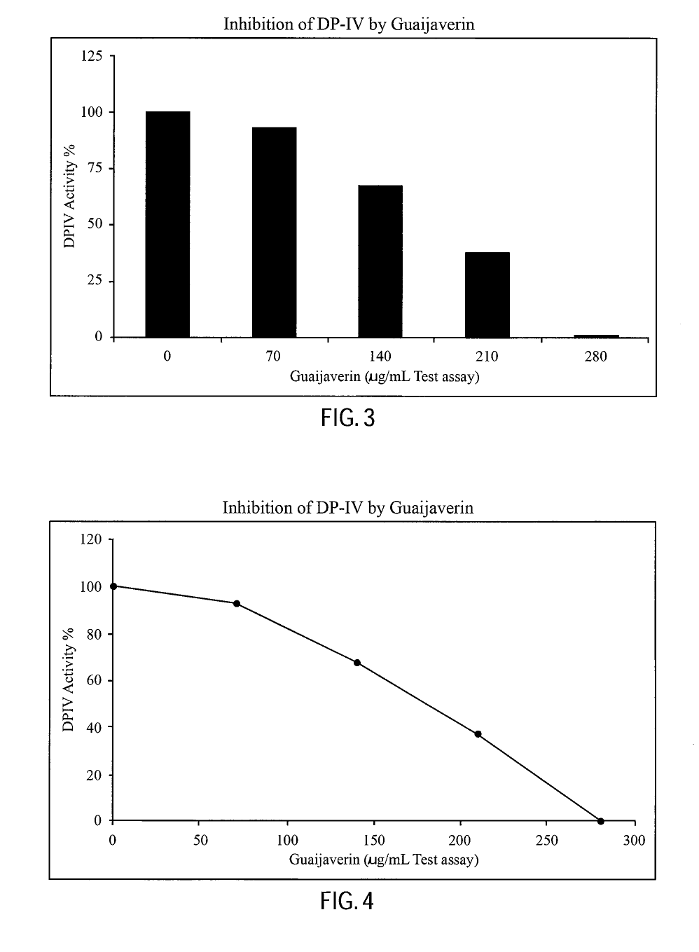

As shown in FIGS. 3 and 4, guaijaverin yields a clear, dose dependent inhibition of DP-IV. In the test assay system chosen, a concentration between 140-210 μg/ml test assay (100-150 μg/assay volume) yielded a DP-IV inhibition of around 50%.

Tests of Guava xtract:

1. Quantitative Data on the Extract Composition

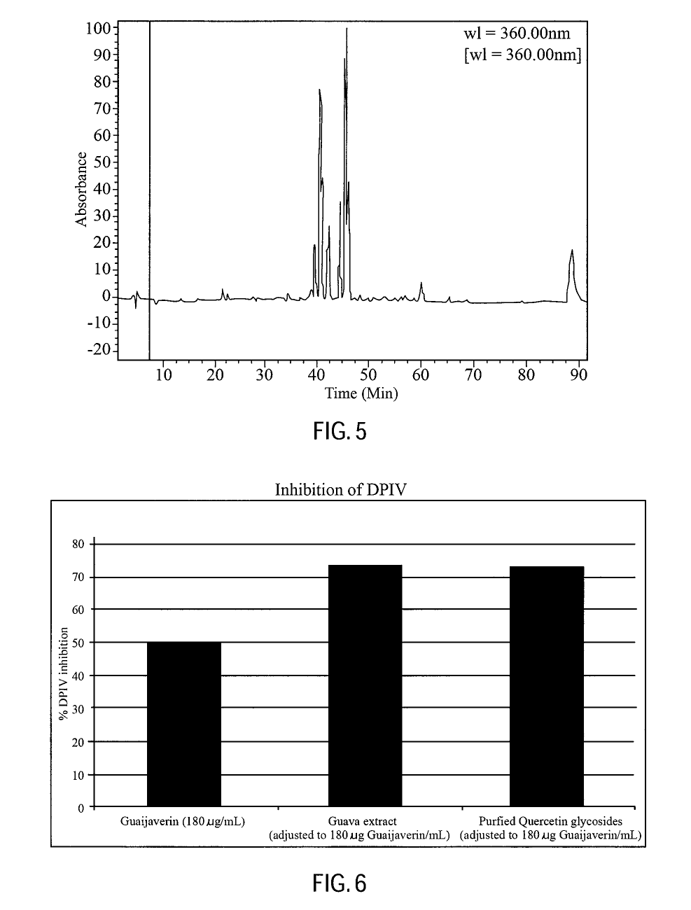

2 g extract/100 ml (as prepared in Example 2) methanolic/aqueous solution (MeOH/H₂O, 50:50, v/v/) were analyzed for the composition of quercetin-glycosides. As shown in FIG. 5, there are 7 main quercetin-glycosides found in the extract. Structures for these peaks were proposed based on MS investigations. For quantification the peak area-% of individual flavonol-glycosides were correlated with the amount of quercetin obtained from the same sample after acidic hydrolysis (corrected for the mass proportion of quercetin in the corresponding structure proposed).

The HPLC conditions for quantitative analysis were as follows:

Ternary gradient pump set to 1.00 ml/min

Mobile Phase A: 0.1% phosphoric acid in water

Mobile Phase B: 0.1% phosphoric acid in acetonitrile

Gradient: 00-15 min: 0-8% B; 15-70 min: 8-24% B; 70-85 min: 24-35% B

Wavelength: 360 nm

Temperature: 30° C.

Injection Vol: 25 μL

Column: Spherisorb ODS 250×4.6 mm

Composition of guava extract derived from HPLC/MS investigations:

| | Structure of Flavonol- | |
|---|---|---|
| Elution order | Glycoside | Content in mg/g extract |
| 1 | Querc-glucoside- | 16.8 |
| | arabinoside | |
| 2 | Querc-hexose | 52.9 |
| 3 | Methyl-Querc-hexose | 53.5 |
| 4 | Querc-glucoside | 19.6 |
| | (isoquercitrin) | |
| 5 | Querc-pentose | 27.7 |
| 6 | Guaijaverin | 65.7 |
| 7 | Querc-pentose | 77.6 |
| Sum | | 313.8 |

In total, each gram of guava extract contained 313.8 mg flavonol-glycosides [equals 31.4% (m/m) quercetin related glycosides]. The content of the lead compound guaijaverin was 65.7 mg/g extract [equivalent to 6.57% (m/m)].

2. Stability of Individual Flavonol-Glycosides During Incubation Conditions

The stability of selected flavonol-glycosides under the test conditions for absorption testing with CaCO2 cells was investigated at a concentration of 2 g extract (from Example 2)/100 ml incubation media.

| | Structure of Flavanol- | Stability 60 min. at 37° C., pH = 7 |
|---|---|---|
| Elution order | Glycoside | [% from time = 0] |
| 1 | Querc-glucoside- | 97.2 |
| | arabinoside | |
| 2 | Querc-hexose | 90.6 |
| 3 | Methyl-Querc-hexose | 100.4 |
| 4 | Querc-glucoside | 98.2 |
| 5 | Querc-pentose | 100.0 |
| 6 | Querc-arabinoside | 98.0 |
| 7 | Querc-pentose | 97.8 |

As seen, all flavonol-glycosides tested were stable under ideal conditions.

3. Investigation of absorption of quercetin-glycosides in CaCO2 cells

Sample concentration: 2 g guava extract (from Example 2)/100 ml incubation media

Incubation conditions: 37° C. for 60 minutes (5 replicate incubations)

Data evaluation: Ratio of peak areas of individual flavonol-glycosides obtained from HPLC analysis of incubation media (immediately after preparation and after 60 minutes of incubation) and the cell content. Results are mean values of 5 incubations.

Uptake (% of flavonol-glycosides present in incubation media)

| | Proposed structure of | |
|---|---|---|
| Elution order | Flavanol-Glycoside | % uptake by CaCO2 cells |
| 1 | Querc-glucoside- | 2.3 |
| | arabinoside | |
| 2 | Querc-hexose | 3.9 |
| 3 | Methyl-Querc-hexose | 4.7 |
| 4 | Querc-glucoside | 4.5 |
| 5 | Querc-pentose | 2.2 |
| 6 | Querc-arabinoside | 5.3 |
| 7 | Querc-pentose | 2.6 |

As seen, the absolute uptake of flavonol glycosides was estimated to between 2.3 and 5.3%. The best absorption was found for quercetin-arabinoside followed closely by quercetin-glucoside and the methyl-quercetin-hexose.

When taking the quantitative composition into consideration, the amount of absorbed quercetin-glycosides sums up to 12 mg/g extract.

Culturing of CaCo-2 Cells

CaCo-2 cells were cultured in Dulbeccos's Modified Eagle Medium containing 20% fetal bovine serum, 1.2% nonessential amino acids, 0.83 mM L-glutamine, 1.2% penicillin-streptomycin and 0.1% mercaptoethanol in an atmosphere of 5% CO₂ and 95% air at 37° C. Cells were grown in 75 cm² culture-flasks (T75) and subcultured after one week (every other day washed with PBS buffer, removed with trypsin and transferred to an new culture flask).

CaCo-2 Test

For experiments, cells were seeded in 6 well plates at a density of 3×10⁵ cells per well and grown in an atmosphere of 5% CO₂ and 95% air at 37° C. 7 to 8 days until confluency was reached. The cells were washed with PBS buffer and incubated with the flavonol for 60 minutes.

After the corresponding incubation time, 900 μl of incubation medium used was taken from each well and mixed with 100 μl formic acid. The cells were washed with PBS buffer and removed using 1 ml of 10% formic acid. Cells were sonicated 3 times for 30 seconds, centrifuged for 10 minutes and the pellets were discarded. The supernatant was used as sample for HPLC.

4 DPI-V Test of Selected Quercetin Glycosides

The activity of guava-extract (From Example 2) towards DP-IV inhibition was compared to guaijaverin and a purified fraction containing all flavonol-glycosides occurring in the guava extract. The purified fraction was prepared by HPLC wherein all fractions that contained active components were combined and concentrated at reduced pressure to afford the purified extract. The purified extract was then dissolved in the incubation medium to a preferred concentration.

All three samples were adjusted to contain 180 μg guaijaverin/ml test assay, a concentration shown to inhibit DP-IV in human plasma by 50%.

FIG. 6 shows the results of this experiment.

180 μg guaijaverin/ml test yielded inhibition of DP-IV by 50%. The guava extract was adjusted to contain 180 μg guaijaverin/ml test assay and was found to inhibit DP-IV by slightly more than 70%. The purified fraction of guava extract containing the entire flavonol-glycoside fraction was adjusted to contain 180 μg guaijaverin/ml test assay and was found to inhibit DP-IV by slightly more than 70%.

These results demonstrated that:

(a) Guaijaverin is one of the active components in terms of DP-IV inhibitory activity.

(b) Other flavonol glycosides present in guava extract exert also DP-IV inhibitory activity and potentiate the effect of guaijaverin.

(c) Based on guaijaverin content, guava extract acts by 50% stronger than pure guaijaverin supporting the synergistic activity of all flavonol glycosides.

(d) The DP-IV inhibitory activity appears to reside in the flavonol-glycoside fraction.

In another experiment, the guava extract (1 g containing 65.7 mg guaijaverin) was diluted to contain 180 μg guaijaverin/ml test assay solution for DP-IV testing. This concentration of guaijaverin (standardized compound) was shown to inhibit DP-IV by 50% in the assay setting chosen.

Testing of the extract at this concentration (2.7 mg/ml test assay solution) yielded an inhibition of 73.2% indicating that the extract contains synergistically acting compounds in addition to guaijaverin.

Based on these results, chromatographic fractions containing the major quercetin related glycosides were fractionated in a preparative scale. The collected fractions containing the major quercetin related glycosides were concentrated by applying vacuum (rotovapor) and adjusted to represent approximately the same amount of quercetin related glycosides as the original extract. When submitting the purified fraction of quercetin related glycosides at the same concentration as they occur in the guava extract, the resulting inhibition of DP-IV was calculated to 72.5%.

The flavonol-glycosides were isolated by preparative chromatography. After collection of the fractions containing the individual flavonol-glycosides, each fraction was evaporated to dryness. The dry residues were collected and the purity was checked by chromatography. Each individual flavonol-glycoside was submitted to DP-IV testing at concentrations between 100-500 μg flavonol-glycoside/ml.

From the test results following concentrations for 50% inhibition of DPIV were estimated.

| | Structure of Flavanol- | Concentration for |
|---|---|---|
| Elution order | Glycoside | 50% inhibition of DPIV [mmol/L] |
| 1 | Querc-glucoside- | 0.80 |
| | arabinoside | |
| 2 | Querc-hexose | 1.20 |
| 3 | Methyl-Querc-hexose | >2 |
| 4 | Querc-glucoside | 0.90 |
| 5 | Querc-pentose | >2 |
| 6 | Querc-arabinoside | 0.40 |
| | (guaijaverin) | |
| 7 | Querc-pentose | >2 |

Although the present invention has been described with reference to preferred embodiments, persons skilled in the art will recognize that changes may be made in form and detail without departing from the spirit and scope of the invention. All references cited throughout the specification, including those in the background, are incorporated herein in their entirety.

Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. An isolated guava extract comprising quercetin related flavonol-glycosides comprising peltatoside and guaijaverin.

2. The isolated guava extract of claim 1, further comprising isoquercetin.

3. The isolated guava extract of claim 1, wherein sesquiterpenes and tannins are substantially removed.

4. The isolated guava extract of claim 3, wherein less than about 5% by weight of the total weight of the extract is sesquiterpenes and tannins.

5. The isolated guava extract of claim 4, wherein less than about 1% by weight of the total weight of the extract is sesquiterpenes and tannins.

6. The isolated guava extract of claim 1, wherein the quercetin related flavonol glycosides comprise at least 30 weight percent of the total weight of the extract.

7. The isolated guava extract of claim 2, wherein sesquiterpenes and tannins are substantially removed.

8. The isolated guava extract of claim 7, wherein less than about 5% by weight of the total weight of the extract is sesquiterpenes and tannins.

9. The isolated guava extract of claim 8, wherein less than about 1% by weight of the total weight of the extract is sesquiterpenes and tannins.

10. The isolated guava extract of claim 2, wherein the quercetin related flavonol glycosides comprise at least 30 weight percent of the total weight of the extract.

11. The isolated guava extract of claim 1, further comprising at least one of quercitin-hexose, methylquercetin-hexose, isoquercitrin, morin-pentose, or quercitrin-pentose.

12. The isolated guava extract of claim 11, wherein sesquiterpenes and tannins are substantially removed.

13. The isolated guava extract of claim 12, wherein less than about 5% by weight of the total weight of the extract is sesquiterpenes and tannins.

14. The isolated guava extract of claim 13, wherein less than about 1% by weight of the total weight of the extract is sesquiterpenes and tannins.

15. The isolated guava extract of claim 11, wherein the quercetin related flavonol glycosides comprise at least 30 weight percent of the total weight of the extract.

16. The isolated guava extract of claim 2, further comprising at least one of quercitin-hexose, methylquercetin-hexose, isoquercitrin, morin-pentose, or quercitrin-pentose.

17. The isolated guava extract of claim 16, wherein sesquiterpenes and tannins are substantially removed.

18. The isolated guava extract of claim 17, wherein less than about 5% by weight of the total weight of the extract is sesquiterpenes and tannins.

19. The isolated guava extract of claim 18, wherein less than about 1% by weight of the total weight of the extract is sesquiterpenes and tannins.

20. The isolated guava extract of claim 16, wherein the quercetin related flavonol glycosides comprise at least 30 weight percent of the total weight of the extract.

21. The isolated guava extract of claim 1, which is capable of inhibiting DP-IV activity or DP-IV like activity.

22. An isolated guava extract obtained from a process comprising extracting guava leaves and/or guava fruit with an alcohol and water at an elevated temperature to obtain a resultant extract; subjecting the resultant extract to reduced pressure to produce an aqueous concentrate; subjecting the concentrate to centrifugation to separate solids; subjecting the clarified solution to a chromatography column and eluting the column with water followed by an alcohol to afford an effluent; subjecting the effluent to reduced pressure to produce a second aqueous concentrate; passing the second aqueous concentrate through a polyamide resin to remove impurities and then desorbing active components with an alcohol; concentrating the active components at reduced pressure to afford the isolated extract.

23. A dipeptidy peptidase IV (DP-IV) inhibitor obtained from a process comprising extracting guava leaves and/or guava fruit with an alcohol and water at an elevated temperature to obtain a resultant extract; subjecting the resultant extract to reduced pressure to produce an aqueous concentrate; subjecting the concentrate to centrifugation to separate solids; subjecting the clarified solution to a chromatography column and eluting the column with water followed by an alcohol to afford an effluent; subjecting the effluent to reduced pressure to produce a second aqueous concentrate; passing the second aqueous concentrate through a polyamide resin to remove impurities and then desorbing active components with an alcohol; concentrating the active components at reduced pressure to afford the DP-IV inhibitor.

24. The DP-IV inhibitor according to claim 23, which is capable of inhibiting DP-IV activity or DP-IV like activity.

25. A method to produce a guava extract capable of inhibiting dipeptidyl peptidase IV activity comprising the steps of:
contacting a guava source of guava leaves and/or guava fruit with an alcohol and water at an elevated temperature to obtain a resultant extract;
subjecting the resultant extract to reduced pressure to produce an aqueous concentrate;
subjecting the aqueous concentrate to centrifugation to separate solids to produce a clarified solution;
subjecting the clarified solution to column chromatography and eluting the column with water followed by elution an alcohol to afford an effluent;
subjecting the effluent to reduced pressure to produce a second aqueous concentrate;
passing the second aqueous concentrate through a polyamide resin to remove impurities and then desorbing active components with an alcohol; and
concentrating the active components at reduced pressure to afford a concentrate.

26. The method of any claim 25, wherein the guava source is from a plant selected from Psidium cattleianum, Psidium cattleianum ssp. Lucidum, Psidium guajava, Psidium guineense, Psidium littorale, Psidium molle or Psidium schiedeanum.

27. The method of claim 26, wherein the hydrophobic column is composed of a crosslinked polystyrene/divinylbenzene copolymer.

28. The method of claim 27, wherein the concentrate is further processed by membrane filtration.

29. A method to treat a disease or condition which is a glucose metabolism disorder, diabetes mellitus, obesity, atherosclerosis, lowering LDL cholesterol, or for relieving conditions associated with women's menstruation comprising the step of administering to an individual in need thereof, a therapeutically effective amount of the composition of claim 1, such that the disease or condition is treated.

30. The method of claim 29, wherein the composition further comprises one or more of isoquercetin, quercitin-hexose, methylquercetin-hexose, morin-pentose, or quercitrin-pentose.

31. The method of claim 29, wherein sesquiterpenes and tannins are substantially removed from the extract.

32. The method of claim 31, wherein less than about 5% by weight of the total weight of the extract is sesquiterpenes and tannins.

33. The method of claim 30, wherein sesquiterpenes and tannins are substantially removed from the extract.

34. The method of claim 33, wherein less than about 5% by weight of the total weight of the extract is sesquiterpenes and tannins.
